SEQUENCE LISTING (22 sequences)

SEQ ID NO: 1 - DNA, 20 bp, Artificial Sequence
  Synthetic polymer.
tccatgacgttcctgatgct

SEQ ID NO: 2 - DNA, 24 bp, Artificial Sequence
  Synthetic polymer.
tcgtcgttttgtcgttttgtcgtt

SEQ ID NO: 3 - DNA, 20 bp, Artificial Sequence
  Synthetic polymer.
ggtgcatcgatgcagggggg

SEQ ID NO: 4 - DNA, 20 bp, Artificial Sequence
  Synthetic polymer.
cgaggcuaucuagaauguac

SEQ ID NO: 5 - DNA, 17 bp, Artificial Sequence
  Synthetic polymer.
guacauucuagauagcc

SEQ ID NO: 6 - DNA, 19 bp, Artificial Sequence
  Synthetic polymer.
gcugcacacgguucagaat

SEQ ID NO: 7 - DNA, 19 bp, Artificial Sequence
  Synthetic polymer.
gcuauuacauugugcauta

SEQ ID NO: 8 - DNA, 19 bp, Artificial Sequence
  Synthetic polymer.
caggaugaguuacuugaaa

SEQ ID NO: 9 - DNA, 19 bp, Artificial Sequence
  Synthetic polymer.
uaaagaaccauggcuaacc

SEQ ID NO: 10 - DNA, 23 bp, Artificial Sequence
  Synthetic polymer.
auucugaaccgugugcagcmumu

SEQ ID NO: 11 - DNA, 23 bp, Artificial Sequence
  Synthetic polymer.
uaaugcacaauguaauagcmumu

SEQ ID NO: 12 - DNA, 21 bp, Artificial Sequence
  Synthetic polymer.
ucaaguaacucauccugmumu

SEQ ID NO: 13 - DNA, 23 bp, Artificial Sequence
  Synthetic polymer.
gguuagccaugguucuuuamumu

SEQ ID NO: 14 - protein, 9 aa, Artificial Sequence
  Synthetic polymer.
FLGPWPAAS

SEQ ID NO: 15 - protein, 10 aa, Artificial Sequence
  Synthetic polymer.
LLALAAGLAV

SEQ ID NO: 16 - protein, 9 aa, Artificial Sequence
  Synthetic polymer.
FLYPFLSHL

SEQ ID NO: 17 - protein, 9 aa, Artificial Sequence
  Synthetic polymer.
ILEYLTAEV

SEQ ID NO: 18 - protein, 9 aa, Artificial Sequence
  Synthetic polymer.
VLAVFIKAV

SEQ ID NO: 19 - protein, 8 aa, Artificial Sequence
  Synthetic polymer.
LSEKLERI

SEQ ID NO: 20 - protein, 10 aa, Artificial Sequence
  Synthetic polymer.
LLLSAEPVPA

SEQ ID NO: 21 - protein, 9 aa, Artificial Sequence
  Synthetic polymer.
ALSRKVAEL

SEQ ID NO: 22 - DNA, 19 bp, Artificial Sequence
  Synthetic polymer.
gcgacugguuacccggucg

(19) United States
(12) Patent Application Publication
(10) Pub. No.: US 2023/0044337 A1
(43) Pub. Date: Feb. 9, 2023

(54) VACCINATION AGAINST ANTIGENS INDUCED IN PATHOGEN-INFECTED CELLS

(71) Applicant: University of Miami, Miami, FL (US)

(72) Inventors: Eli GILBOA, Miami, FL (US); Greta GARRIDO, Miami, FL (US); Brett SCHRAND, Miami, FL (US)

(21) Appl. No.: 17/759,139

(22) PCT Filed: Jan. 28, 2021
(86) PCT No.: PCT/US21/15457

§ 371 Date: Jul. 20, 2022

Related U.S. Application Data
(60) Provisional application No. 62/967,152, filed on Jan. 29, 2020.

(51) Int. Cl. A61K 31/7088; A61K 39/12; A61K 47/54; A61P 31/12

(52) U.S. Cl. CPC A61K 31/7088; A61K 39/12; A61K 47/543; A61P 31/12; A61K 2039/55561

(57) ABSTRACT

The present invention relates, in part, to methods of generating immune responses in subjects to treat an infectious disease.

### CROSS-REFERENCE TO RELATED APPLICATIONS

This application claims the benefit of and priority to U.S. Provisional Patent Application No. 62/967,152 filed Jan. 29, 2020, the contents of which are hereby incorporated by reference in their entirety.

### FIELD OF THE INVENTION

The present invention relates, in part, to methods for generating immune responses for anti-infective uses.

### SEQUENCE LISTING

The contents of the text file submitted electronically herewith are incorporated herein by reference in their entirety. A computer readable format copy of the Sequence Listing (filename: SEB-005PC_Sequence_Listing_ST25, date recorded: Jan. 27, 2021; file size: 13,000 bytes).

### BACKGROUND

Infectious diseases remain a major health concern. Case in point are viruses belonging to the Herpesviridae family, such as cytomegalovirus (CMV), Epstein-Barr virus (EBV), and herpes simplex viruses (HSV), that represent an important unmet clinical need. For example, CMV is the major cause of mortality in solid organ and allogeneic hematogenous stem cell transplantation. Despite broad efforts in academia and industry, with the exception of several varicella vaccine formulations to prevent herpes zoster, no vaccines against CMV, HSV or EBV have been approved so far for clinical use. By way of further example, human immunodeficiency virus (HIV) has seen success in decreasing HIV-associated morbidity and mortality (e.g. with combination antiretroviral therapy) but patients afflicted with HIV have shorter life expectancy than those without the virus, and the underlying causes are probably multifactorial, including premature aging, drug toxicities, and comorbidities. There is a need for new approaches to fight infections.

### SUMMARY OF THE INVENTION

Accordingly, the present invention provides methods of altering the immune system of a subject that has a pathogen-infected cell. For instance, the present methods stimulate an immune response, e.g., a vaccine response, against cell-encoded antigens that are experimentally/therapeutically induced in the pathogen-infected cell. In various embodiments, the present methods induce antigens in a pathogen-infected cell and, accordingly, a subject's immune response can be directed to such cell. In various aspect, the present methods vaccinate against transporter associated with antigen processing (TAP) downregulation-induced antigens in any pathogen-infected cell.

In an aspect, the present invention provides a method of treating an pathogenic infection in a subject need thereof, comprising administering an effective amount of an immune-modulating agent to pathogen-infected cells in the subject to direct a subject's existing immune response to cell-encoded antigens that are experimentally/therapeutically induced in the pathogen-infected cell, where the immune-modulating agent inhibits and/or downregulates a mediator of antigen processing and induces antigen formation; and the subject has an existing immune response against the induced antigen.

In embodiments, the pathogen is bacterial, viral antigen, or parasitic. In embodiments, the pathogen is viral. In embodiments, the virus is from the Herpesviridae family, optionally selected from cytomegalovirus (CMV), Epstein-Barr virus (EBV), and herpes simplex viruses (HSV) or is a retrovirus, optionally selected from human immune deficiency (HIV) and simian immune deficiency (SIV).

In embodiments, the immune-modulating agent elicits and/or boosts an anti-pathogenic immune response, e.g. elicits and/or boosts an immune response against cell-encoded antigens that are experimentally/therapeutically induced in a pathogen-infected cell. In embodiments, the immune-modulating agent inhibits and/or downregulates a mediator of an antigen processing pathway. In embodiments, the immune-modulating agent inhibits and/or downregulates one or more of a mediator of ER aminopeptidase associated with antigen processing (ERAAP), transporter associated with antigen processing (TAP), and invariant chain (Ii). In embodiments, the immune-modulating agent comprises an oligonucleotide molecule, such as a small interfering RNA, or a micro RNA, or an antisense RNA directed against the mediator of antigen processing or a gene-editing protein directed against the mediator of antigen processing, the gene-editing protein selected from a Clustered Regularly Interspaced Short Palindromic Repeats (CRISPR), TALEN, nickase, and zinc finger protein. In embodiments, immune-modulating agent further comprises a targeting agent. In embodiments, targeting agent is oligonucleotide aptamer ligand, a protein-based targeting agent (optionally an antibody), peptide, or a combination thereof. In embodiments, the immune-modulating agent is targeted to the pathogen-infected cells or a target cell, optionally being a dendritic cell or other antigen presenting cell.

In embodiments, the method reduces the severity or duration of the pathogenic infection.

In embodiments, the pathogenic infection is CMV and the subject has a compromised immune system, optionally due to stem cell or organ transplants and/or an HIV infection. In embodiments, the pathogenic infection is CMV and the subject is a newborn infected with CMV before birth (i.e. afflicted with congenital CMV), an infant (i.e. afflicted with perinatal CMV), or a pregnant woman.

In embodiments, the pathogenic infection is EBV and the subject is afflicted with infectious mononucleosis.

In embodiments, the pathogenic infection is HSV, selected from HSV-1 and HSV-2.

In embodiments, the pathogenic infection is HIV and the subject is afflicted with stage 1 HIV infection, stage 2 HIV infection, stage 3 HIV infection, an opportunistic infection or disease, or AIDS.

In embodiments, the immune-modulating agent is delivered to the subject via a lipid carrier.

In embodiments, the present methods further comprise administering an additional therapeutic agent.

### BRIEF DESCRIPTION OF THE DRAWINGS

FIG. 1A-D. CpG-TAP siRNA pulsed DC stimulate human PBMC derived CD8+ T cells in vitro that recognize tumor cells with reduced TAP expression. FIG. 1A. Time course of humanTAP-1 RNA levels in DC treated with CpG-TAP siRNA. Monocyte derived human DC were treated with CpG-Ctrl or TAP siRNAs and at indicated time points mRNA was generated and quantified by qRT-PCR. Shown are means and SEM performed in duplicates. Results from two independent experiments were combined. FIG. 1B. Presentation of p14 a TAP deficiency-induced peptide in 518A2 melanoma cells treated with Nucl-siRNAs and cultured with a cognate CD8+ T cell clone that recognized the HLA-A2-p14 complex(32). IFN gamma production after 20 h stimulation was measured by ELISA. Means and SEM of quadruplicate wells (n=2). FIG. 1C. Stimulation of TAP TEIPP specific CD8+ T cells. CD8+ T cells from an HLA-A2 donor were stimulated with autologous DC treated with CpG-TAP siRNA. After two rounds of stimulation, CD8+ T cells were isolated and cocultured with TAP deficient 518A2 cells (518A2 TAP KO) or with TAP-sufficient parental cells (518A2) treated with Nucl-siRNAs. IFN gamma production after 20 h stimulation was measured by ELISA. Shown are means and SEM of quadruplicate wells (n=2). FIG. 1D. Polyclonality of the TAP TEIPP specific CD8+ T cells. CD8+ T cell cultures as described in panel C were incubated with 518A2 cells pulsed with six previously described HLA-A2 restricted TAP-deficiency-induced peptides(32). MAGE peptide was used as negative control. IFN gamma production after 20 h stimulation was measured by ELISA. Shown are means and SEM of quadruplicate wells (n=2).

FIG. 2 In vitro “vaccination” with CpG-TAP siRNA against TAP downregulation-induced antigens presented by CMV and EBV infected cells. MRC5 and Ramos or two human cell lines susceptible to infection with CMV or EBV, respectively. Antigen specific recognition of the infected cells by the CD8+ T cells was determined by measuring IFN gamma secretion. Evidence that the CD8+ T cells recognized TAP downregulation-induced antigens is indicated by the fact that the PBMC-derived T cells cultured with CpG conjugated to control siRNA did not result in IFN gamma secretion. For each labelled condition there are two bars: CpG-Ctrl (left) and CpG-TAP (right).

FIG. 3 In vitro “vaccination” with CpG-TAP siRNA against antigens induced in HIV infected cells by TAP downregulation. Experimental protocol as described in FIG. 1 except that the cultured CD8+ T cells are reacted with the human CEM174 T cell line infected with the NL4-3 HIV virus which are incubated with the broadly neutralizing anti-HIV env 2G12 antibody conjugated to a TAP or control siRNA. TAP+ and TAP− 518A2 cells are human melanoma tumor cell lines that serve as positive and negative control. For each labelled condition there are two bars: CpG-Ctrl (left) and CpG-TAP (right).

### DETAILED DESCRIPTION

The present invention provides methods of altering the immune system of a subject that is has a pathogen-infected cell to stimulate an immune response, e.g., a vaccine response, against the infecting pathogens. In various embodiments, the present methods induce antigens in a pathogen-infected cell and, accordingly, a subject's immune response is directed to such cell.

In an aspect, the present invention provides a method of treating an pathogenic infection in a subject need thereof, comprising administering an effective amount of an immune-modulating agent to pathogen-infected cells in the subject to direct a subject's existing immune response against cell-encoded antigens that are experimentally/therapeutically induced in a pathogen-infected cell, where the immune-modulating agent inhibits and/or downregulates a mediator of antigen processing and induces antigen formation; and the subject has an existing immune response against the induced antigen.

Methods of Infection Treatment

In some aspects, the methods are used to eliminate pathogens. In some aspects, the present methods are used to treat one or more infections. In some embodiments, the present invention provides methods of treating viral infections (including, for example, HIV and HCV), parasitic infections (including, for example, malaria), and bacterial infections. In various embodiments, the infections induce immunosuppression. For example, HIV infections often result in immunosuppression in the infected subjects. Accordingly, the treatment of such infections may involve, in various embodiments, modulating the immune system to favor immune stimulation. Alternatively, the present invention provides methods for treating infections that induce immunoactivation. For example, intestinal helminth infections have been associated with chronic immune activation. In these embodiments, the treatment of such infections may involve modulating the immune system to favor immune inhibition over immune stimulation.

In various embodiments, the present invention provides methods of treating viral infections including, without limitation, acute or chronic viral infections, for example, of the respiratory tract, of papilloma virus infections, of herpes simplex virus (HSV) infection, of human immunodeficiency virus (HIV) infection, and of viral infection of internal organs such as infection with hepatitis viruses. In some embodiments, the viral infection is caused by a virus of family Flaviviridae. In some embodiments, the virus of family Flaviviridae is selected from Yellow Fever Virus, West Nile virus, Dengue virus, Japanese Encephalitis Virus, St. Louis Encephalitis Virus, and Hepatitis C Virus. In other embodiments, the viral infection is caused by a virus of family Picornaviridae, e.g., poliovirus, rhinovirus, coxsackievirus. In other embodiments, the viral infection is caused by a member of Orthomyxoviridae, e.g., an influenza virus. In other embodiments, the viral infection is caused by a member of Retroviridae, e.g., a lentivirus. In other embodiments, the viral infection is caused by a member of Paramyxoviridae, e.g., respiratory syncytial virus, a human parainfluenza virus, rubulavirus (e.g., mumps virus), measles virus, and human metapneumovirus. In other embodiments, the viral infection is caused by a member of Bunyaviridae, e.g., hantavirus. In other embodiments, the viral infection is caused by a member of Reoviridae, e.g., a rotavirus. In other embodiments, the viral infection is caused by a member of the Herpesviridae family, e.g., cytomegalovirus (CMV), Epstein-Barr virus (EBV), and herpes simplex viruses (HSV)

In various embodiments, the present invention provides methods of treating parasitic infections such as protozoan or helminths infections. In some embodiments, the parasitic infection is by a protozoan parasite. In some embodiments, the oritiziab parasite is selected from intestinal protozoa, tissue protozoa, or blood protozoa.

Illustrative protozoan parasites include, but are not limited to, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Plasmodium vivax, Plasmodium ovale, Plasmodium malariae, Plasmodium falciparum, Trichomonas vaginalis, and Histomonas meleagridis. In some embodiments, the parasitic infection is by a helminthic parasite such as nematodes (e.g., Adenophorea). In some embodiments, the parasite is selected from Secementea (e.g., Trichuris trichiura, Ascaris lumbricoides, Enterobius vermicularis, Ancylostoma duodenale, Necator americanus, Strongyloides stercoralis, Wuchereria bancrofti, Dracunculus medinensis). In some embodiments, the parasite is selected from trematodes (e.g. blood flukes, liver flukes, intestinal flukes, and lung flukes). In some embodiments, the parasite is selected from: Schistosoma mansoni, Schistosoma haematobium, Schistosoma japonicum, Fasciola hepatica, Fasciola gigantica, Heterophyes, Paragonimus westermani. In some embodiments, the parasite is selected from cestodes (e.g., Taenia solium, Taenia saginata, Hymenolepis nana, Echinococcus granulosus).

In various embodiments, the present invention provides methods of treating bacterial infections. In various embodiments, the bacterial infection is by a gram-positive bacteria, gram-negative bacteria, aerobic and/or anaerobic bacteria. In various embodiments, the bacteria is selected from, but not limited to, Staphylococcus, Lactobacillus, Streptococcus, Sarcina, Escherichia, Enterobacter, Klebsiella, Pseudomonas, Acinetobacter, Mycobacterium, Proteus, Campylobacter, Citrobacter, Neisseria, Bacillus, Bacteroides, Peptococcus, Clostridium, Salmonella, Shigella, Serratia, Haemophilus, Brucella and other organisms. In some embodiments, the bacteria is selected from, but not limited to, Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophila, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

Immune-Modulating Agents

In various embodiments, the present invention pertains to an immune-modulating agent. In various embodiments, the immune-modulating agent elicits and/or boosts an anti-infective immune response. In various embodiments, the immune-modulating agent is a vaccine. In various embodiments, the immune-modulating agent stimulates the generation of an immune response against neoantigens. In various embodiments, the immune-modulating agent vaccinates against a neoantigen. In various embodiments, the immune-modulating agent elicits and/or boosts an anti-infective immune response via generation of a neoantigen-mediated immune response.

In some embodiments, the immune-modulating agent induces neoantigens in pathogen-infected cells in situ.

In some embodiments, the immune-modulating agent provides targeted inhibition and/or downregulation of key mediators of antigen processing pathways. In various embodiments, the immune-modulating agent provides targeted inhibition and/or downregulation of ERAAP. In various embodiments, the immune-modulating agent provides targeted inhibition and/or downregulation of transporter associated with antigen processing (TAP). In various embodiments, the immune-modulating agent provides targeted inhibition and/or downregulation of invariant chain (Ii).

In some embodiments, the immune-modulating agent provides targeted inhibition and/or downregulation of key mediators of antigen processing pathways, e.g., one or more of ERAAP, TAP, and Ii, and provides the same epitopes in the cells having the inhibition and/or downregulation (i.e. the epitope generation is not stochastic).

In some embodiments, the immune-modulating agent provides targeted inhibition and/or downregulation of key mediators of antigen processing pathways to pathogen-infected cells.

ERAAP is an ER-resident aminopeptidase that trims the TAP-transported peptides to optimize their association with the nascent MHC class I molecules (see Nature, 2002; 419(6906):480-3). Importantly, without wishing to be bound by theory, ERAAP deficiency induces significant alterations in the MHC class I presented peptidome. Some peptides are lost while new peptides appear, the latter probably, without wishing to be bound by theory, because they escape ERAAP processing. Like TAP-deficient cells. ERAAP-deficient cells are immunogenic in wild type mice inducing T cell response against the new ERAAP-loss induced peptides to which the wild type mouse has not been tolerized, and inhibit tumor growth. The new peptides are presented both by classical MHC class Ia molecules as well as by nonclassical MHC class Ib molecules, specifically Qa-1b. A dominant peptide presented by Qa-1b in the H-2b background was identified as FYAEATPML (FL9) derived from FAM49B protein).

Qa-1b restricted presentation of the FL9 peptide stimulates CD8+ T cell responses in wild type mice that can kill ERAAP-deficient, but not ERAAP sufficient, targets.

TAP is a critical component of MHC class I presentation responsible for transporting the proteasome generated peptides from the cytoplasm to the ER where they are loaded onto the nascent MHC class I molecules (see Nat Rev Immunol. 2011; 11(12):823-36.) TAP function is frequently downregulated in tumors conceivably, without wishing to be bound by theory, to avoid immune recognition. TAP-deficient cells present novel peptide-MHC complexes resulting from alternative antigen processing pathways that are upregulated or become dominant in the absence of the canonical TAP-mediated pathway. TAP deficiency-induced peptides, referred to as “T cell epitopes associated with impaired peptide processing” or TEIPP, are presented by classical MHC class Ia molecules as well as by nonclassical Qa-1b molecules. Importantly, TAP-deficient cells or DC loaded with TEIPP peptide restricted to both the classical MHC Ia and Qa-1b can stimulate CD8+ T cell responses in wild type mice and vaccination with TEIPP loaded DC, TAP-deficient DC, or adoptive transfer of TEIPP specific CDB+ T cells was shown to inhibit the growth of TAP-deficient, but not TAP sufficient, tumors.

Invariant chain is a polypeptide involved in the formation and transport of MHC class II protein. The cell surface form of the invariant chain is known as CD74. MHC class II's path toward the cell surface involves, in the rough endoplasmic reticulum, an association between the alpha and beta chains and a Ii, which stabilizes the complex. Without the invariant chain, the alpha and beta proteins will not associate. Ii trimerizes in the ER, associates with MHC class II molecules and is released from the ER as a nine subunit complex. This MHC-invariant complex passes from the RER to, and out of, the Golgi body. Before moving to the cell surface, the vesicle containing this complex fuses with an endocytic compartment where an external protein has been broken into fragments. Here the invariant chain is proteolytically degraded and a peptide from the external protein associates with the MHC II molecule in the channel between the alpha-1 and beta-1 domains. The resulting MHC II-peptide complex proceeds to the surface where it is expressed.

In some embodiments, the immune-modulating agent inhibits and/or downregulates a nonsense-mediated mRNA (NMD) process. NMD is an evolutionarily conserved surveillance mechanism in eukaryotic cells that prevents the expression of mRNAs containing a premature termination codon (PTC). Without wishing to be bound by theory, inhibition of results in the upregulation of several products encoded by the PTC-containing mRNAs and many of these products, resulting from aberrant splicing or NMD-dependent autoregulated alternative splicing encode new peptides that have not induced tolerance. In some embodiments, the immune-modulating agent is a small interfering RNA (siRNA) which downregulates certain NMD factors (e.g., SMG1, UPF1, UPF2, UPF3, RENT1, RENT2, eIF4A, UPF1, UPF2, UPF3B, RNPS1, Y14, MAGOH, NMD1, or combinations thereof).

In some embodiments, the immune-modulating agent comprises a small interfering RNA, or a micro RNA, or an antisense RNA.

In some embodiments, the immune-modulating agent comprises a oligonucleotide molecule, such as a small interfering RNA, or a micro RNA, or an antisense RNA which is targeted to pathogen-infected cells or a target cell, optionally being a dendritic cell or other antigen presenting cell, e.g., by a targeting agent.

In some embodiments, the immune-modulating agent comprises a oligonucleotide molecule, such as a small interfering RNA, or a micro RNA, or an antisense RNA which is targeted to pathogen-infected cells or a target cell, optionally being a dendritic cell or other antigen presenting cell by conjugation to an oligonucleotide aptamer ligand or a protein-based or peptide-based targeting agent.

In some embodiments, the targeting strategy for a pathogen-infected cell involves ligands (e.g. antibodies, peptides, antibodies) that bind to pathogen (e.g. viral) products expressed on the surface of pathogen-infected cells. In some embodiments, the targeting strategy for a professional antigen presenting cell involves targeting to different receptors on the cell surface than what is used for pathogen-infected cell, inclusive of, by way of non-limiting example, TLR9 and Clec9a, using strategies like, by way of non-limiting example, CpG oligonucleotides.

In various embodiments, the immune-modulating agent produces inhibition and/or downregulation of specific mediators of an antigen processing pathway like one or more of ERAAP, TAP, and Ii and stimulates novel epitopes to which the immune system has not been tolerized and thereby they could function essentially as neoantigens. Such epitopes are non-mutated subdominant epitopes that are normally not presented and therefore carry a reduced risk of autoimmunity. Importantly, epitopes generated by downregulation of one or more of ERAAP, TAP, and Ii are not generated as a result of random events in the cell, therefore they are more like to be shared, namely the same epitope presented by any cell in which the corresponding target was downregulated.

In various embodiments, the immune-modulating agent does not substantially trigger an autoimmune reaction.

In various embodiments, the immune-modulating agent comprises a targeting agent which is specific for a desired target cell, e.g., a pathogen-infected cell (e.g., a cell infected by any of the pathogens or microorganisms described herein). In various embodiments, the immune-modulating agent comprises a targeting agent which is specific for a desired target cell, e.g., a dendritic cell or other antigen presenting cell.

In embodiments, a CpG oligonucleotide is used to target TAP siRNA to dendritic cells or other antigen presenting cell.

In embodiments, the targeting agent is directed to a protein, antigen, or receptor on a dendritic cell or other antigen presenting cell, such as, for example, CLEC9A, DEC205, XCR1, RANK, CD36/SRB3, LOX-1/SR-E1, CD68, MARCO, CD163, SR-A1/MSR, CD5L, SREC-1, CL-PI/COLEC12, SREC-II, LIMPIIISRB2, RP105, TLR4, TLR1, TLR5, TLR2, TLR6, TLR3, TLR9, 4-IBB Ligand/TNFSF9, IL-12/IL-23 p40, 4-Amino-1,8- naphthalimide, ILT2/CD85j, CCL21/6Ckine, ILT3/CD85k, 8-oxo-dG, ILT4/CD85d, 8D6A, ILT5/CD85a, A285, lutegrin a 4/CD49d, Aag, Integrin β 2/CD18, AMICA, Langerin, B7-2/CD86, Leukotriene B4 RI, B7-H3, LMIR1/CD300A, BLAME/SLAMF8, LMIR2/CD300c, Clq R1/CD93, LMIR3/CD300LF, CCR6, LMIR5/CD300LB CCR7, LMIR6/CD300LE, CD40/TNFRSF5, MAG/Siglec-4-a, CD43, MCAM, CD45, MD-1, CD68, MD-2, CD83, MDL-1/CLEC5A, CD84/SLAMF5, MMR, CD97, NCAMLI, CD2F-10/SLAMF9. Osteoactivin GPNMB, Chem 23, PD-L2, CLEC-1, RP105, CLEC-2, CLEC-8, Siglec-21CD22, CRACC/SLAMF7, Siglec-3/CD33, DC-SIGN, DCE205, Siglec-5, DC-SIGNR/CD299, Siglec-6, DCAR, Siglec-7, DCIR/CLEC4A, Siglec-9, DEC-205, Siglec-10, Dectin-1/CLEC7A, Siglec-F, Dectin-2/CLEC6A, SIGNR1/CD209, DEP-1/CD148, SIGNR4, DLEC, SLAM, EMMPRIN/CD147, TCCR/WSX-1, Fc-γ R1/CD64, TLR3, Fc-γ RIIB/CD32b, TREM-1, Fc-γ RIIC/CD32c, TREM-2, Fc-Y RIIA/CD32a, TREM-3, Fc-γ RII/CD16, TREML1I/TLT-1, ICAM-2/CD102 and Vanilloid R1, with, e.g. antibodies, peptides, or aptamers.

In embodiments, the targeting agent is directed to a receptor on a dendritic cell or other antigen presenting cell, such as Clec9a or DEC205, with, e.g. antibodies, peptides, or aptamers.

In various embodiments, the immune-modulating agent comprises a targeting agent such as an aptamer-oligonucleotide molecule. In some embodiments, the aptamer is specific for a desired target cell, e.g., a pathogen-infected cell (e.g., a cell of any of the pathogens or microorganisms described herein). In various embodiments, the immune-modulating agent comprises a nucleolin aptamer. In various embodiments, the immune-modulating agent comprises an epithelial cell adhesion molecule (EpCAM) aptamer (e.g., 5′-GCGACUGGUUACCCGGUCG-3′ (SEQ ID NO: 22) or variations thereof). In various embodiments, the immune-modulating agent comprises a VEGF aptamer.

In various embodiments, the targeting agent is an antibody, antibody format, or paratope-comprising fragment thereof directed against the protein, antigen, or receptor of interest. In various embodiments, the antibody is a full-length multimeric protein that includes two heavy chains and two light chains. Each heavy chain includes one variable region (e.g., V_{H}) and at least three constant regions (e.g., CH₁, CH₂ and CH₃), and each light chain includes one variable region (V_{L}) and one constant region (C_{L}). The variable regions determine the specificity of the antibody. Each variable region comprises three hypervariable regions also known as complementarity determining regions (CDRs) flanked by four relatively conserved framework regions (FRs). The three CDRs. referred to as CDR1, CDR2, and CDR3, contribute to the antibody binding specificity. In some embodiments, the antibody is a chimeric antibody. In some embodiments, the antibody is a humanized antibody.

In some embodiments, the targeting agent is an antibody derivative or format. In some embodiments, the targeting agent comprises a targeting moiety which is a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; a peptide aptamer; an alterases; a plastic antibodies; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)₂, a peptide mimetic molecule, or a synthetic molecule, as described in US patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 20101119446, and/or U.S. Pat. No. 7,166,697, the contents of which are hereby incorporated by reference in their entireties. See also, Storz MAbs. 2011 May-June; 3(3): 310-317.

In some embodiments, the antibody is conjugated with an oligonucleotide molecule.

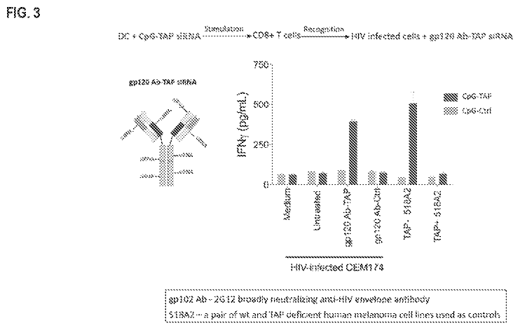

In some embodiments, the antibody is conjugated with siRNAs. Such antibodies can be constructed by, e.g., “decorating” the antibody with 6-8 copies of a short oligonucleotide and then hybridizing the siRNA to the antibody via a short complementary sequence engineered on the siRNA. The end product is an antibody targeting multiple copies of siRNA to the HIV infected cell (see diagram in FIG. 3). Such an antibody is, in various embodiments, used in the methods of the present invention.

In some embodiments, the antibody is targeted to the viral envelope protein (e.g. env or gp120) that is expressed in the HIV infected cell. In some embodiments, the antibody is one or more broadly neutralizing antibodies against one or more HIV antigens,

In some embodiments, the targeting agent is a peptide directed to a cell or marker of interest.

In various embodiments, the oligonucleotide molecule comprises at least one of a short interfering RNA (siRNA); a micro-interfering RNA (miRNA); antisense oligonucleotides; a small, temporal RNA (stRNA); a short, hairpin RNA (shRNA), and antisense RNA, or combinations thereof. In various embodiments, the oligonucleotide molecule targets specific mediators of an antigen processing pathway like one or more of ERAAP, TAP, and Ii.

In various embodiments, the immune-modulating agent comprises a molecule suitable for RNA interference, i.e. the process of sequence-specific post-transcriptional gene silencing in animals mediated by short interfering RNAs (siRNAs). In various embodiments, the immune-modulating agent comprises a siRNA.

The presence of long dsRNAs in cells stimulates the activity of a ribonuclease III enzyme referred to as dicer. Dicer is involved in the processing of the dsRNA into short pieces of dsRNA known that are siRNAs. siRNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNAs duplex. Accordingly, some embodiments of the invention contemplate use of dsRNA to downregulate protein expression from mRNA.

In various embodiments, the present siRNA are between about 18-30 basepairs (e.g., about 18, or about 19, or about 20, or about 21, or about 22, or about 23, or about 24, or about 25, or about 26, or about 27, or about 28, or about 29, or about 30 basepairs) and induce the RNA interference (RNAi) pathway. In some embodiments, the siRNAs are 21mers with a central 19 bp duplex region and symmetric 2-base 3′-overhangs on the termini, although other variations of length and overhang are possible.

The strands of a double-stranded interfering RNA (e.g., a siRNA) may be connected to form a hairpin or stem-loop structure (e.g., a shRNA). Thus, the dsRNA of some embodiments of the invention may also be a hairpin or short hairpin RNA (shRNA).

In various embodiments, the immune-modulating agent comprises a miRNA. MiRNAs are short nucleic acid molecules that are able to regulate the expression of target genes. See review by Carrington et al. Science, Vol. 301(5631):336-338, 2003. MiRNAs are often between about 18 to about 24 nucleotides in length. MiRNAs act as repressors of target mRNAs by promoting their degradation, when their sequences are perfectly complementary, and/or by inhibiting translation, when their sequences contain mismatches. Without being bound by theory, mature miRNAs are believed to be generated by pol II or pol III and arise from initial transcripts termed—miRNAs. These pri-miRNAs are frequently several thousand bases long and are therefore processed to make much shorter mature miRNAs. These pri-miRNAs may be multicistronic and result from the transcription of several clustered sequences that organize what may develop into many miRNAs. The processing to yield miRNAs may be two-steps. First, pri-miRNAs may be processed in the nucleus by the RNase Drosha into about 70- to about 100-nucleotide hairpin-shaped precursors (pre-miRNAs). Second, after transposition to the cytoplasm, the hairpin pre-miRNAs may be further processed by the RNase Dicer to produce a double-stranded miRNA. The mature miRNA strand may then be incorporated into the RNA-induced silencing complex (RISC), where it may associate with its target mRNAs by base-pair complementarity and lead to suppression of protein expression. The other strand of the miRNA duplex that is not preferentially selected for entry into a RISC silencing complex is known as the passenger strand or minor miRNA or star (*) strand. This strand may be degraded. It is understood that, unless specified, as used herein an miRNA may refer to pri- and/or pre- and/or mature and/or minor (star) strand and/or duplex version of miRNA.

In various embodiments, the immune-modulating agent comprises an antisense oligonucleotide. An antisense oligonucleotide is a nucleic acid strand (or nucleic acid analog) that is complementary to an mRNA sequence. Antisense occurs naturally and can trigger RNA degradation by the action of the enzyme RNase H. In various embodiments, the antisense oligonucleotide is non-naturally occurring. In various embodiments, the antisense oligonucleotide comprises one or more nucleic acid analogs. In various embodiments, the antisense oligonucleotide is nuclease resistant and activates RNase H. In various embodiments, the antisense oligonucleotide comprises phosphorothioate RNA and other nucleic acid analogs that bind to RNA and sterically inhibit processes without activating RNase H (such as 2′-O-methyl phosphorothioate RNA, Morpholino oligos, locked nucleic acids, or peptide nucleic acids). These latter RNase-H independent oligos do not trigger degradation of mRNA but they can be to block translation, alter splicing of pre-mRNA, inhibit activity of miRNA, block ribozyme activity, and interfere with various other processes that require some other factor to bind to a particular sequence on an RNA molecule.

In various embodiments, the immune-modulating agent is one of US Patent Publication No. 2012/0263740, the entire contents of which are hereby incorporated by reference.

In some embodiments, the oligonucleotide molecule and/or targeting agent, such as a aptamer, has one or more nucleotide substitutions (e.g., at least one of adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)alkynylcytosine, 5-fluorouracil, 5-bromouracil, pseudoisocytosine, 2-hydroxy-5-methyl-4-triazolopyridin, isocytosine, isoguanin, inosine, non-naturally occurring nucleobases, locked nucleic acids (LNA), peptide nucleic acids (PNA), variants, mutants, analogs or combinations thereof.

In various embodiments, the aptamer and/or the siRNA (e.g., the sense strand) comprise fluoro-modified pyrimidines, e.g., 2′-fluoro-modified pyrimidines, e.g., one or more of 2′-fluoro-cytosine (C), 2′-fluoro-thymine (T), and 2′-fluoro-uracil (U).

In some embodiments, any immune-modulating agent (and/or additional agents) described herein is formulated in accordance with procedures as a composition adapted for a mode of administration described herein.

In some embodiments, the present invention provides vaccination with neoantigen mRNA-lipid nanocarriers. In some embodiments, vaccination with mRNA complexed to lipid carriers like DOPE and DOTMA can be undertaken (Nature, 2016; 534(7607):396-401). Illustrative lipid carriers include 1,2-Dioleoyl-sn-glycero-3-phosphatidylcholine (DOPC), 1,2-Dioleoyl-sn-glycero-3-phosphatidylethanolamine (DOPE), cholesterol, N-[1-(2,3-Dioleyloxy)propyl]N,N,N-trimethylammonium chloride (DOTMA), 1,2-Dioleoyloxy-3-trimethylammonium-propane (DOTAP), Dioctadecylamidoglycylspermine (DOGS), N-(3-Aminopropyl)-N,N-dimethyl-2,3-bis(dodecyloxy)-1-propanaminium bromide (GAP-DLRIE), cetyltrimethylammonium bromide (CTAB), 6-lauroxyhexyl ornithinate (LHON), 1-)2,3-Dioleoloxypropyl)2,4,6-trimethylpyridinium (2Oc), 2,3-Dioleyloxy-N-[2(sperminecarboxamido)-ethyl]-N,N-dimethyl-1-propanaminium trifluoroacetate (DOSPA), 1,2-Dioleyl-3-trimethylammonium-propane (DOPA), N-(2-Hydroxyethyl)-N,N-dimethyl-2,3-bis(tetradecyloxy)-1-propanaminium bromide (MDRIE), Dimyristooxypropyl dimethyl hydroxyethyl ammonium bromide (DMRI), 3β-[N—(N′,N′-Dimethylaminoethane)-carbamoyl]cholesterol (DC-Chol), Bis-guanidium-tren-cholesterol (BGTC), 1,3-Dioleoxy-2-(6-carboxy-spermyl)-propylamide (DOSPER), Dimethyloctadecylammonium bromide (DDAB), Dioctadecylamidoglicylspermidin (DSL), rac-[(2,3-Dioctadecyloxypropyl)(2-hydroxyethyl)]-dimethylammonium chloride (CLIP-1), rac-[2(2,3-Dihexadecyloxypropyl-oxymethyloxy)ethyl]trimethylammonium chloride (CLIP-6), Ethyldimyrisotylphosphatidylcholine (EDMPC), 1,2-Distearyloxy-N,N-dimethyl-3-aminopropane (DSDMA), 1,2-Dimyistoyl-trimethylammoniumpropane (DMTAP), O,O′-Dimyristyl-N-lysyl asparate (DMKE), 1,2-Distearoyl-sn-glycero-3-ethylphosphocholine (DSEPC), N-Palmitoyl-D-erythro-spingosyl carbamoyl-spermine (CCS), N-t-Butyl-No-tetradecyl-3-tetradecylaminopropionamidine (diC14-amidine), Octadecenolyoxy[ethyl-2-heptadecenyl-3 hydroxyethyl]imidazolinium chloride (DOTIM), N1-Cholesteryloxycarbonyl-3,7-diazanonane-1,9-diamine (CDAN) and 2-(3-[Bis-(3-amino-propyl)-amino]propylamino)-N-ditetradecylcarbamoylme-ethyl-acetamide (RPR2091290). In some embodiments, this approach will be used to vaccinate against neoantigens using total RNA, mRNA enriched poly A+ RNA, or amplified polyA+ RNA from syngeneic fibroblasts or B cells as described above.

Routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In some embodiments, the administering is effected orally or by parenteral injection.

Any immune-modulating agent (and/or additional agents) described herein can be administered parenterally. Such immune-modulating agents (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

Subjects

In various embodiments, the subject is afflicted with a chronic infection. In various embodiments, the subject is afflicted with one of hepatitis B, hepatitis C, and human papilloma viruses. In various embodiments, the subject is afflicted with H. pylori bacteria.

A depressed immune system, such as can be found in HIV-positive or AIDS subjects, transplant recipients, geriatric subjects and so forth, can be another criterion for selecting suitable subjects.

In various embodiments, the subject is not afflicted with cancer and/or is not susceptible to becoming afflicted with cancer.

In embodiments, the pathogenic infection is CMV and the subject has a compromised immune system, optionally due to stem cell or organ transplants and/or an HIV infection. In embodiments, the pathogenic infection is CMV and the subject is a newborn infected with CMV before birth (i.e. afflicted with congenital CMV), an infant (i.e. afflicted with perinatal CMV), or a pregnant woman.

In embodiments, the pathogenic infection is EBV and the subject is afflicted with infectious mononucleosis.

In embodiments, the pathogenic infection is HIV and the subject is afflicted with stage 1 HIV infection, stage 2 HIV infection, stage 3 HIV infection, an opportunistic infection or disease, or AIDS.

The term subject, as used herein unless otherwise defined, is a mammal, e.g., a human. Experimental animals are also included, such as a mouse, rat, guinea pig, dog, cat, horse, cow, pig, or non-human primate, such as a monkey, chimpanzee, or baboon. In one embodiment, the subject is a veterinary patient, including the animals described herein. In one embodiment, the subject is a human.

The method also can be practiced in entirely healthy subjects who are not known to be at risk.

In various embodiments, there is provided co-administration of the present immune modulating agent with one or more additional therapeutic agents. Such co-administration does not require the therapeutic agents to be administered to the subject by the same route of administration. Rather, each therapeutic agent can be administered by any appropriate route, for example, parenterally or non-parenterally. Further, co-administration relates to simultaneous or sequential administration.

In some embodiments, the immune modulating agent described herein acts synergistically when co-administered with an additional therapeutic agent. In such embodiments, the immune modulating agent and the additional therapeutic agent may be administered at doses that are lower than the doses employed when the agents are used in the context of monotherapy.

Further, in various embodiments, the present methods relate to treating a subject who has previously undergone treatment with an additional therapeutic agent. Further, in various embodiments, the present methods relate to treating a subject who is presently undergoing treatment with an additional therapeutic agent.

In some embodiments, inclusive of, without limitation, infectious disease applications, the present invention pertains to anti-infectives as additional agents. In some embodiments, the anti-infective is an anti-viral agent including, but not limited to, Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, and Foscarnet. In some embodiments, the anti-infective is an anti-bacterial agent including, but not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem). In some embodiments, the anti-infectives include anti-malarial agents (e.g., chloroquine, quinine, mefloquine, primaquine, doxycycline, artemether/lumefantrine, atovaquone/proguanil and sulfadoxine/pyrimethamine), metronidazole, tinidazole, ivermectin, pyrantel pamoate, and albendazole.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g, for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

This invention is further illustrated by the following non-limiting examples.

### EXAMPLES

### Example 1: Proof of Concept of Antigen Development in Tumor Cells

The present inventors have described a new vaccination concept targeting new antigens that are experimentally induced in the tumor cell and dendritic cell by targeted downregulation of the peptide transporter TAP using a corresponding siRNA. In this proof-of-concept study, a broad spectrum nucleolin binding aptamer (Nucl) was used to target the TAP siRNA to tumor cells that “decorates” the tumor cells with new antigens and a CpG oligonucleotide to target the TAP siRNA to dendritic cells (DC) that elicits an immune response against the induced antigens.

It was shown that the concept is translatable to human settings, by showing that in vitro enrichment of PBMC derived CD8+ T cells for induced antigen specificities (by incubating the T cells with DC treated with CpG-TAP siRNA) recognizes tumor cells in culture provided the tumor cells were treated with Nucl-TAP siRNA to present the induced antigens.

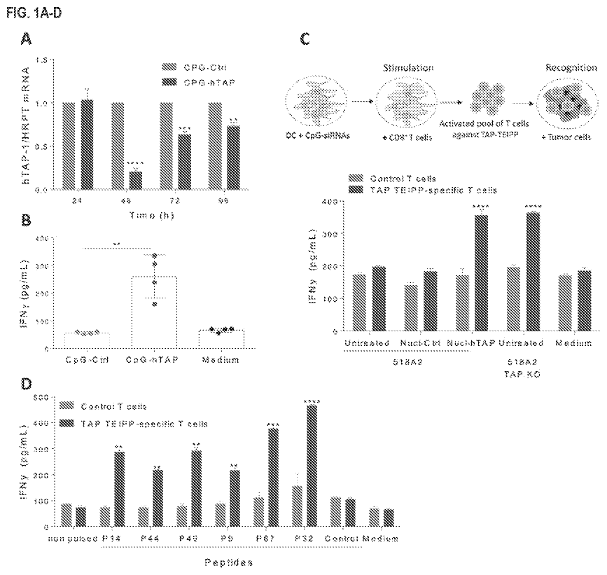

Specifically, FIG. 1A-D shows that in vitro stimulated TAP T cell epitopes associated with impaired peptide processing (TEIPP) specific T cells recognize TAP^{low} human tumor cells. To determine whether vaccination against TAP TEIPP could be applicable to human patients, the inventors tested if dendritic cells (DC) pulsed with CpG-TAP siRNA are capable of stimulating in vitro CD8+ T cells that will recognize tumor cells treated with Nucl-TAP siRNA. Human monocyte derived DC treated with the CpG ODN conjugated to a human TAP specific siRNA led to the partial downregulation of TAP mRNA (FIG. 1A), and presentation of p14 to a cognate T cell clone (FIG. 1B). FIG. 1C shows that CpG-TAP siRNA treated DC stimulate autologous CD8+ T cells which recognized both TAP-deficient as well as Nucl-TAP, but not Nucl-Ctrl, siRNA treated TAP-sufficient tumor cells. Cells that downregulate TAP present multiple epitopes mostly derived from housekeeping products. FIG. 1D shows that the CpG-TAP siRNA stimulated CD8+ T cells recognized DC pulsed with HLA-A2 restricted peptides that are presented by TAP deficient tumor cells. This suggests that CpG-TAP siRNA treated DC can stimulate a polyclonal CD8+ T cell response against multiple shared TAP TEIPP presented also by TAP-deficient tumor cells, and thereby could enhance the recognition of a broad range of tumors with reduced TAP expression.

In this Example, the inventors have demonstrated, inter alia, that it is possible to “mark” tumor cells with (TAP downregulation-induced) new antigens to make them more “visible” to the immune system and hence more susceptible to vaccination.

### Example 2: Antigen Induction in Pathogen-Infected Cells

In this Example, the inventors, inter alia, establish a protocol, by which CpG-TAP siRNA is used to vaccinate against TAP downregulation-induced antigens in any pathogen-infected cell—provided TAP can be specifically and only downregulated in the infected cell. That is, the Inventors target TAP downregulation to infected cells—unless, the virus does it itself.

Experiments were carried out in culture with human cells because human viruses that do not replicate in mouse cells were used. The experimental protocol parallels the protocol described in FIG. 1A-D for human tumors. In brief, the first step was to enrich for CD8+ T cells with specificities to TAP downregulation-induced antigens by culturing PBMC derived CD8+ T cells with autologous DC treated with CpG-TAP siRNA which stimulates the proliferation of TAP downregulation-induced antigen specific T cells.

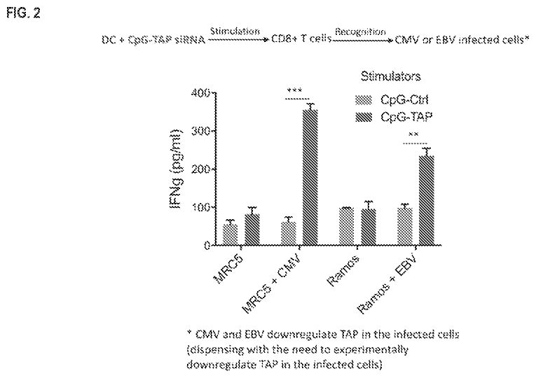

First, vaccination against pathogens belonging to the Herpesviridae family, CMV, EBV or HSV was attempted. Viruses belonging to this family downregulate TAP during acute infection as one of several mechanisms they employ to evade immune elimination, thereby dispensing with the need to experimentally downregulate TAP in the infected cells. The experiment in FIG. 2 shows that CMV or EBV infected cells are recognized by CD8+ T cells enriched for specificities to TAP downregulation-induced antigens (by incubating PBMC-derived CD8+ T cells with DC+CpG-TAP siRNA. These experiments, therefore suggest that vaccination with CpG-TAP siRNA could elicit protective immunity also against pathogens belonging to the Herpesviridae family that downregulate TAP in the infected cells.

Second, vaccination against pathogens against HIV was attempted. Unlike Herpesviridae, HIV infection does not result in TAP downregulation. In this instance, therefore, the Inventors needed to downregulate TAP in the infected cells. A question was how to target the TAP siRNA specifically and only to HIV infected cells. The inventors targeted to the viral envelope protein (env or gp120) that is expressed in the HIV infected cell; in effect “marking” cells infected with HIV. This was initially done using an HIV gp120 env aptamer but has also been accomplished using antibodies that offer advantages in terms of recognizing a broad range of HIV species. The approach of conjugating siRNAs to the antibody involves first “decorating” the antibody with 6-8 copies of a short oligonucleotide and then hybridizing the siRNA to the antibody via a short complementary sequence engineered on the siRNA. The end product is an antibody targeting multiple copies of siRNA to the HIV infected cell (see diagram in FIG. 3). The experiment shown in FIG. 3 is similar to the experiment described in FIG. 2 (and FIG. 1) except that the cultured CD8+ T cells enriched for specificities to induced antigens are reacted with HIV infected cell, recognize HIV infected cells but only if they are treated with a gp 120Ab-TAP siRNA conjugate.

Accordingly, CpG-TAP siRNA stimulated CD8+ T cells recognize tumor cells as well as pathogen-infected cells in which TAP expression is reduced, naturally as is the case with Herpesviridae or HPV transformed tumor cells, or experimentally by targeting siRNA to tumor cells or infected cells, respectively.

Materials & Methods for Examples 1 and 2

Cells and Culture Conditions

Ramos and MRC-5 cells were purchased from ATCC.

Cell lines were cultured in RPMI-1640 medium (A20, 4T1, 67NR, Caski, C33A, Ramos, TMD8, TC-1, B6 HLF and DC2.4 cells), Dulbecco's modified Eagle's medium (MC38, MRC-5, SW480 and SW620) or Iscove's Modified Dulbecco's Medium (RMA, RMA-S, 518A2 and mouse T cell activation assays) from Gibco, supplemented with 8-10% heat-inactivated FCS, 100 U/ml penicillin, and 100 μg/ml streptomycin. Mouse T cells were additionally supplemented with 1 mM sodium pyruvate, 0.05 mM β-mercaptoethanol, and 2 mM minimal essential medium (MEM) non-essential amino acids. TC-1 and B6 HLF cells were additionally supplemented with 1 mM sodium pyruvate, 2 mM minimal essential medium (MEM) non-essential amino acids, and 50 μg/ml gentamycin. For TC-1 cells also was added 0.4 mg/ml G418, and 0.2 mg/ml hygromycin. DC and T cell culture media from Stemcell were used for human DC differentiation and T cell culture, respectively. All cell lines and assay cultures were maintained at 37° C. and 5% CO2. All cells were tested regularly for mycoplasma contamination.

Design of CpG-siRNA Conjugates

Sequences of CpG ODNs used in the study were as follows CpG 1668 (5′-tccatgacgttcctgatgct-3 SEQ ID NO: 1), CpG 2006 (5′-tcgtcgttttgtcgttttgtcgtt-3′ SEQ ID NO: 2) and CpG D19 (5′-ggTGCATCGATGCAGggggg-3′ SEQ ID NO: 3). Bases in capital letters are phosphodiester, bases in lower case are phosphorothioate (nuclease resistant). These sequences were extended at the 3′ end with the following sequence (termed linker): 5′ CGAGGCUAUCUAGAAUGUAC (SEQ ID NO: 4), and were purchased from Trilink Biotechnologies. Nucleolin aptamer, extended at the 3′ end with the following sequence (termed linker): 5′ GUACAUUCUAGAUAGCC (SEQ ID NO: 5), were purchased from Trilink Biotechnologies. Complementary linker sequences extending from the sense strand of murine TAP2 (5′GCUGCACACGGUUCAGAAT SEQ ID NO: 6), murine ERAAP (5′GCUAUUACAUUGUGCAUTA SEQ ID NO: 7), human TAP1 (5′ CAGGAUGAGUUACUUGAAA SEQ ID NO: 8) or control (Ctrl) (5′ UAAAGAACCAUGGCUAACC SEQ ID NO: 9) siRNAs were ordered from IDT and contained 2′ O-methyl modified pyrimidines with the last two bases being deoxynucleotides. Antisense siRNA sequences, ordered from IDT, were as follows: murine TAP2 (5′ AUUCUGAACCGUGUGCAGCmUmU SEQ ID NO: 10), murine ERAAP (5′ UAAUGCACAAUGUAAUAGCmUmU SEQ ID NO: 11), human TAP1 (5′ UUUCAAGUAACUCAUCCUGmUmU SEQ ID NO: 12) and Ctrl (5′ GGUUAGCCAUGGUUCUUUAmUmU SEQ ID NO: 13) whereby ‘m’ indicated the presence of a 2′ O′-methyl modified ribonucleotide. CpGs or Nucleolin aptamer were annealed to duplex siRNAs in PBS at 82° C. for four min or 37° C. for 10 min respectively, in a block heater and allowed to cool to room temperature.

siRNA Knockdown and qPCR Analysis

For in vitro siRNA knockdown, cells were plated in triplicates onto 24-well plates (2.5-5×10⁴ cells) for 18 h. After complete adhesion, cells were incubated with 0.5 μM of Nucl-siRNA or 0.3 uM of CpG-siRNAs conjugates two times every 8 h. Cells were harvested 24, 48, 72 or 96 h, after the last treatment. For in vivo siRNA knockdown, Balb/c mice were injected once subcutaneously with CpG-siRNAs (0.75 nmol) close to inguinal LN in the right flank. LN were excised 24 h later and DC cells were isolated using CD11 MicroBeads (Miltenyi Biotec). Murine TAP-2 or human TAP-1 mRNA was quantified by qPCR. RNA was isolated using an RNeasy kit (QIAGEN). RNA was quantified using an Agilent 2100 Bioanalyzer (Agilent Technologies). cDNA synthesis was performed using the High Capacity cDNA Reverse Transcription kit (Applied Biosystems). cDNA equivalents of 25-50 ng of mRNA were used per reaction in a TaqMan qPCR assay using the Step One qPCR machine (Applied Biosystems), with primer sets corresponding to the gene of interest or housekeeping products.

In Vitro Virus Infection

MRC-5 cells were plated in a 6-well plate and infected at MOI of 5 with the TB40/E hCMV strain that had 2.2×10\hat{ }8 PFU/ml. Cells were incubated for 90 min and media was replaced. After 48 h, cells were split into 96-well plates. Co-culture with polyclonal T cell pool was started at 72 h post infection after determining HLA-ABC downregulation via flow cytometry. Likewise, Ramos cells were spinoculated with EBV virus at MOI=100 at RT for 90 min at 800×g and placed in a T25 flask. Cells were taken out and cocultured with polyclonal T cells at 48 h after determining HLA-ABC downregulation.

Generation of Human CD8+ T Cells Enriched for TAP Downregulation-Induced Epitopes.

Human DC differentiated from monocytes were incubated with 0.3 uM of CpG-siRNAs conjugates two times every 24 h. Twenty-four hours after second pulse, DC were cocultured with homologous CD8+ T cells in presence of IL2 (20 ng/ml) and IL-15 (50 ng/ml) for 6 days. A third pulse with CpG-siRNAs was done at day off of coculture. Culture medium was replenished every 2-3 d with fresh complete T cell medium with cytokines. After two rounds of specific stimulation, the CD8+ T cells were isolated using positive selection CD8+ T cell isolation kit (Miltenyi Biotec).

Recognition of Targets by Human TAP Downregulation-Induced Epitope Specific CD8+ T Cells.

CpG-siRNAs or TAP-siRNAs treated, peptide pulsed, virus-infected or untreated cells were cocultured with activated Lnb5 T cells, 1A8 T cells, or TAP deficiency epitope enriched CD8+ T cells (E:T ratio, 1:10). Peptides (1 μg/ml) were purchased from Anaspec and sequences were as follow P14-FLGPWPAAS (SEQ ID NO: 14); P29-LLALAAGLAV (SEQ ID NO: 15); P44-FLYPFLSHL (SEQ ID NO: 16); P49-ILEYLTAEV (SEQ ID NO: 17); P9-VLAVFIKAV (SEQ ID NO: 18); P67-LSEKLERI (SEQ ID NO: 19); P32-LLLSAEPVPA (SEQ ID NO: 20); control MAGE-ALSRKVAEL (SEQ ID NO: 21). Murine or human IFN gamma production after 20 h stimulation was measured by ELISA from R&D systems. Cytotoxic activity was determined in 4 h in vitro lactate dehydrogenase assay (Thermo Fisher Scientific). Percentage of specific lysis was calculated as: ([experimental release−effector cell release−spontaneous release][maximum release−spontaneous release])×100.

Statistical Analysis

When variables studied were normally distributed, statistical analysis of multiple comparisons was performed using one-way ANOVA with Tuckey post-test, and comparisons between just two groups were performed using Students' unpaired t test. Nonparametrical methods were applied for not normally distributed variables. For these statistical analyses, multiple comparisons was performed using Kruskall-Wallis with Dunn post-test, and comparisons between just two groups were performed using Mann-Whitney U test. Significance of overall survival was determined via Kaplan-Meier analysis with log-rank analysis. All statistical analyses were performed with Graphpad Prism 6 and 7 (GraphPad). Error bars show standard error of the mean (SEM), and p<0.05 was considered statistically significant. * indicates p<0.05, **p<0.01, ***p<0.001, and ****p<0.0001 unless otherwise indicated. ns denotes not significant.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

1. A method of treating an pathogenic infection in a subject need thereof, comprising administering an effective amount of an immune-modulating agent to pathogen-infected cells in the subject to direct a subject's existing immune response against cell-encoded antigens that are induced in a pathogen-infected cell, wherein:
the immune-modulating agent inhibits and/or downregulates a mediator of antigen processing and induces antigen formation; and
the subject has an existing immune response against the induced antigen.

2. The method of claim 1, wherein the pathogen is bacterial, viral antigen, or parasitic.

3. The method of claim 1, wherein the pathogen is viral.

4. The method of claim 3, wherein the virus is from the Herpesviridae family, optionally selected from cytomegalovirus (CMV), Epstein-Barr virus (EBV), and herpes simplex viruses (HSV) or is a retrovirus, optionally selected from human immune deficiency (HIV) and simian immune deficiency (SIV).

5. The method of claim 1, wherein the immune-modulating agent elicits and/or boosts an immune response against cell-encoded antigens induced in pathogen-infected cells.

6. The method of claim 1, wherein the immune-modulating agent inhibits and/or downregulates a mediator of an antigen processing pathway.

7. The method of claim 1, wherein the immune-modulating agent inhibits and/or downregulates one or more of a mediator of ERAAP, transporter associated with antigen processing (TAP), and invariant chain (ii).

8. The method of claim 1, wherein the immune-modulating agent comprises an oligonucleotide molecule, such as a small interfering RNA, or a micro RNA, or an antisense RNA directed against the mediator of antigen processing or a gene-editing protein directed against the mediator of antigen processing, the gene-editing protein selected from a Clustered Regularly Interspaced Short Palindromic Repeats (CRISPR), TALEN, nickase, and zinc finger protein.

9. The method of claim 1, wherein the immune-modulating agent further comprises a targeting agent.

10. The method of claim 9, wherein the targeting agent is oligonucleotide aptamer ligand, a protein-based targeting agent, peptide, or a combination thereof.

11. The method of claim 1, wherein the immune-modulating agent is targeted to the pathogen-infected cell or a target cell, optionally being a dendritic cell or other antigen presenting cell.

12. The method of claim 1, wherein the method reduces the severity or duration of the pathogenic infection.

13. The method of claim 1, wherein the pathogenic infection is CMV and the subject has a compromised immune system, optionally due to stem cell or organ transplants and/or an HIV infection.

14. The method of claim 1, wherein the pathogenic infection is CMV and the subject is a newborn infected with CMV before birth, an infant, or a pregnant woman.

15. The method of claim 1, wherein the pathogenic infection is EBV and the subject is afflicted with infectious mononucleosis.

16. The method of claim 1, wherein the pathogenic infection is HSV, selected from HSV-1 and HSV-2.

17. The method of claim 1, wherein the pathogenic infection is HIV and the subject is afflicted with stage 1 HIV infection, stage 2 HIV infection, stage 3 HIV infection, an opportunistic infection or disease, or AIDS.

18. The method of claim 1, wherein the immune-modulating agent is delivered to the subject via a lipid carrier.

19. The method of claim 1, further comprising administering an additional therapeutic agent.

20. A method of treating a viral infection in a subject need thereof, comprising administering an effective amount of an immune-modulating agent to viral-infected cells in the subject to direct a subject's existing immune response against cell-encoded antigens that are induced in the viral-infected cells, wherein:
the immune-modulating agent comprises an oligonucleotide molecule and inhibits and/or downregulates one or more of a mediator of ERAAP, transporter associated with antigen processing (TAP), and invariant chain (h) and
the subject has an existing immune response against the induced antigen.
